(19) United States
(12) Patent Application Publication
(10) Pub. No.: US 2020/0214681 A1
(43) Pub. Date: Jul. 9, 2020

(54) GRATING LOBE REDUCTION FOR ULTRASOUND IMAGES AND ASSOCIATED DEVICES, SYSTEMS, AND METHODS

(71) Applicant: KONINKLIJKE PHILIPS N.V., Eindhoven (NL)

(72) Inventors: Seungsoo KIM, ANDOVER, MA (US); Francois Guy Gerard Marie VIGNON, ANDOVER, MA (US); Jun Seob SHIN, MEDFORD, MA (US); Jean-luc Francois-Marie ROBERT, CAMBRIDGE, MA (US); David HOPE SIMPSON, BOTHELL, WA (US); Andrew HANCOCK, SACRAMENTO, CA (US)

(21) Appl. No.: 16/733,050

(22) Filed: Jan. 2, 2020

Related U.S. Application Data
(60) Provisional application No. 62/789,279, filed on Jan. 7, 2019.

(51) Int. Cl. A61B 8/08; A61B 8/00; A61B 8/12; A61B 8/06; G06T 7/00

(52) U.S. Cl. CPC A61B 8/5269; A61B 8/5207; A61B 8/461; A61B 8/12; G06T 2207/30104; A61B 8/06; A61B 8/0883; G06T 7/0012; G06T 2207/10132; A61B 8/488

(57) ABSTRACT

Systems, methods, and devices for generating ultrasound images that have reduced or minimized grating lobe artifacts are provided. In one embodiment, an ultrasound imaging system includes an array of acoustic elements, and a processor in communication with the array. The processor is configured to receive electrical signals from the array, determine, based on the directivity of the acoustic elements, a portion of the electrical signals corresponding to on-axis components, generate a weighting mask based on the portion, and apply the weighting mask to an original image to generate a grating lobe minimized image. Because grating lobe artifacts are caused, in part, by reflections from off-axis objects, applying the weighting mask to an original image can reduce the presence of grating lobe artifacts in the original image.

### CROSS-REFERENCE TO PRIOR APPLICATIONS

This application claims the benefit of U.S. Provisional Patent Application No. 62/789,279, filed Jan. 7, 2019, the contents of which are incorporated herein by reference.

### TECHNICAL FIELD

The present disclosure relates generally to ultrasound imaging and, in particular, to generating grating-lobe-reduced ultrasound images. For example, an ultrasonic medical imaging device can include an array of acoustic elements configured to obtain ultrasound data, the array being in communication with a processor configured to process the obtained ultrasound data based on a directivity of the acoustic elements of the array.

### BACKGROUND

Intravascular ultrasound (IVUS) imaging is widely used in interventional cardiology as a diagnostic tool for assessing a diseased vessel, such as an artery, within the human body to determine the need for treatment, to guide the intervention, and/or to assess its effectiveness. An IVUS device including one or more ultrasound transducers is passed into the vessel and guided to the area to be imaged. The transducers emit ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a cross-sectional image of the vessel where the device is placed.

Solid-state (also known as synthetic-aperture) IVUS catheters are one of the two types of IVUS devices commonly used today, the other type being the rotational IVUS catheter. Solid-state IVUS catheters carry a scanner assembly that includes an array of ultrasound transducers distributed around its circumference along with one or more integrated circuit controller chips mounted adjacent to the transducer array. The controllers select individual acoustic elements (or groups of elements) for transmitting an ultrasound pulse and for receiving the ultrasound echo signal. By stepping through a sequence of transmit-receive pairs, the solid-state IVUS system can synthesize the effect of a mechanically scanned ultrasound transducer but without moving parts (hence the solid-state designation). Since there is no rotating mechanical element, the transducer array can be placed in direct contact with the blood and vessel tissue with minimal risk of vessel trauma. Furthermore, because there is no rotating element, the electrical interface is simplified. The solid-state scanner can be wired directly to the imaging system with a simple electrical cable and a standard detachable electrical connector, rather than the complex rotating electrical interface required for a rotational IVUS device.

In IVUS imaging, a clinical goal is reducing ultrasound image artifacts, such as artifacts produced by grating lobes. Grating lobe artifacts, which appear as blurry, off-axis duplicates of on-axis objects, are particularly common in ultrasound images that are spatially-undersampled. Spatially-undersampled images can result from arrays that do not satisfy the Nyquist sampling criterion, which requires that the pitch, or spacing between acoustic elements in the array, be smaller than half the center wavelength. Given the frequencies at which IVUS imaging devices operate, it may be difficult to manufacture IVUS imaging arrays with acoustic elements and spacings that are small enough to satisfy the Nyquist criterion.

### SUMMARY

Embodiments of the present disclosure provide systems, methods, and devices for generating ultrasound images that have reduced or minimized grating lobe artifacts. In particular, the present disclosure proposes utilizing a calculated amplitude response for an aperture of an array-based ultrasound imaging device in order to determine the signal contributions from different angles of arrival. By determining the fraction or portion of a signal that is provided from on-axis objects, a weighting mask can be generated to reduce or minimize the influence of off-axis signals on the image. Because grating lobe artifacts are caused, in part, by reflections from off-axis objects, applying the weighting mask to an original image can reduce the presence of grating lobe artifacts in the original image.

According to one embodiment, an ultrasound imaging system includes an array of acoustic elements configured to transmit ultrasound energy into a patient's anatomy and receive echoes associated with the transmitted ultrasound energy, the acoustic elements receiving the echoes according to a directivity of the acoustic elements, and a processor in communication with the array. The processor is configured to receive, from the array, electrical signals corresponding to the received echoes, generate an image based on the received electrical signals, determine a portion of the received electrical signals corresponding to on-axis components, wherein the portion is determined based on the directivity of the acoustic elements and the received electrical signals, generate a weighting mask based on the determined portion, apply the weighting mask to the image to generate a grating lobe minimized image, and output, to a display in communication with the processor, the grating lobe minimized image.

In some embodiments, the ultrasound imaging system includes an intravascular ultrasound (IVUS) imaging catheter, and the array of acoustic elements is positioned around a circumference of the IVUS imaging catheter. In some embodiments, the processor is configured to control the array by activating a plurality of apertures, and the processor is configured to determine the portion of the received electrical signals corresponding to the on-axis components by determining an amplitude response of each aperture for the on-axis components of the received electrical signals, and decomposing the amplitude of the electrical signals into a plurality of amplitude basis vectors, wherein the amplitude basis vectors are created based on the directivity of the acoustic elements. In some aspects, the processor is configured to determine the directivity of the acoustic elements based on an angular position of the acoustic elements in the array and an acceptance angle between the acoustic elements and a target position. In other aspects, the processor is configured to determine the directivity of the acoustic elements based further on ultrasound image data. In still other aspects, the processor is configured to generate the weighting mask by generating a weight vector of M elements, wherein the M elements of the weight vector correspond to signal contributions from M different angles of arrival. In some embodiments, the processor is configured to generate the weighting mask by identifying an element of the weight vector that corresponds to an angle of arrival of 0°. In some embodiments, the processor is configured to apply the weighting mask to the image by multiplying the image with the weighting mask.

In another embodiment, a method for ultrasound imaging includes controlling, by a processor, an array of acoustic elements in communication with the processor to transmit ultrasound energy into a patient's anatomy and receive echoes associated with the transmitted ultrasound energy, wherein the acoustic elements receive the echoes according to a directivity of the acoustic elements, receiving, at the processor, electrical signals corresponding to the received echoes, generating an image based on the received electrical signals, determining a portion of the image data corresponding to on-axis components based on the directivity of the acoustic elements and the received electrical signals, generating a weighting mask based on the determined portion, applying the weighting mask to the image to generate a grating lobe minimized image, and outputting, to a display in communication with the processor, the grating lobe minimized image.

In some embodiments, controlling the array to transmit ultrasound energy comprises controlling an intravascular ultrasound (IVUS) imaging catheter, wherein the array of acoustic elements is positioned around a circumference of the IVUS imaging catheter. In some embodiments, controlling the array to transmit the ultrasound energy and to receive the echoes comprises activating a plurality of apertures, and wherein determining the portion of the received electrical signals corresponding to on-axis components comprises determining an amplitude response of each aperture for the on-axis components of the received electrical signals, and decomposing an amplitude of the electrical signals into a plurality of amplitude basis vectors, wherein the amplitude basis vectors are created based on the directivity of the acoustic elements. In some aspects, determining the directivity of the acoustic elements comprises determining the directivity of each acoustic element based on an angular position of the acoustic element in the array and an acceptance angle between the acoustic element and a target position. In other aspects, determining the directivity of the acoustic elements further comprises determining the directivity of the acoustic elements based on ultrasound image data. In some embodiments, generating the weighting mask comprises generating a weight vector of M elements, wherein the M elements of the weight vector correspond to signal contributions from M different angles of arrival. In some embodiments, generating the weighting mask further comprises identifying an element of the weight vector that corresponds to an angle of arrival of 0°. In some embodiments, applying the weighting mask to the image comprises multiplying the image with the weighting mask.

According to another embodiment of the present disclosure, an ultrasound imaging system includes an array of acoustic elements configured to transmit ultrasound energy into a patient's anatomy and receive echoes associated with the transmitted ultrasound energy, and a processor in communication with the array. The processor is configured to generate image data corresponding to the received echoes, generate an image based on the image data, decompose the image data into a plurality of amplitude basis vectors corresponding to different angles of arrival, generate a weighting mask based on the plurality of amplitude basis vectors, generate a grating lobe minimized image by applying the weighting mask to the image, and output, to a display in communication with the processor, the grating lobe minimized image.

In some embodiments, the processor is configured to generate the weighting mask by determining, based on the plurality of amplitude basis vectors, a portion of the image data corresponding to on-axis components, wherein the portion is determined based on a directivity of the acoustic elements of the array and the image data. In some embodiments, the directivity of the acoustic elements is determined based on an angular position of the acoustic elements in the array and an acceptance angle between the acoustic elements and a target position. In some embodiments, the directivity of the acoustic elements is determined based on ultrasound data.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic schematic view of an intraluminal imaging system, according to aspects of the present disclosure.

FIG. 2 is a diagrammatic view of the top of a scanner assembly in a flat configuration, according to aspects of the present disclosure.

FIG. 3 is a diagrammatic perspective view of the scanner assembly shown in FIG. 2 in a rolled configuration around a support member, according to aspects of the present disclosure.

FIG. 4 is a diagrammatic cross-sectional side view of a scanner assembly in a rolled configuration around a support member, according to aspects of the present disclosure.

FIG. 5 is a diagrammatic graphical view of an ultrasound pulse sequence, according to aspects of the present disclosure.

FIG. 6A is a diagrammatic schematic view of an array of acoustic elements receiving reflected ultrasonic energy from an on-axis target object, according to aspects of the present disclosure.

FIG. 6B is a diagrammatic schematic view of an array of acoustic elements receiving reflected ultrasonic energy from an off-axis target object, according to aspects of the present disclosure.

FIG. 7 is a diagrammatic graphical view of a theoretically-derived amplitude response of an aperture, according to aspects of the present disclosure.

FIG. 8 is a diagrammatic graphical view of an empirically-measured amplitude response of an aperture, according to aspects of the present disclosure.

FIG. 9 is a diagrammatic graphical view of the amplitude response shown in FIG. 7 modified using data obtained by the measured amplitude response shown in FIG. 8, according to aspects of the present disclosure.

FIG. 10 is a flow diagram illustrating a method for generating a grating lobe minimized ultrasound image, according to aspects of the present disclosure.

FIG. 11 is a graph of three amplitude responses corresponding to different angles of arrival, according to aspects of the present disclosure.

FIG. 12A is an original, pre-scan-conversion IVUS image of a vessel and a stent, according to aspects of the present disclosure.

FIG. 12B is a grating lobe minimized, pre-scan-conversion IVUS image generated from the original IVUS image shown in FIG. 12A, according to aspects of the present disclosure.

FIG. 13A is a scan-converted version of the IVUS image shown in FIG. 12A, according to aspects of the present disclosure.

FIG. 13B is a scan-converted version of the grating lobe minimized IVUS image shown in FIG. 12B, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

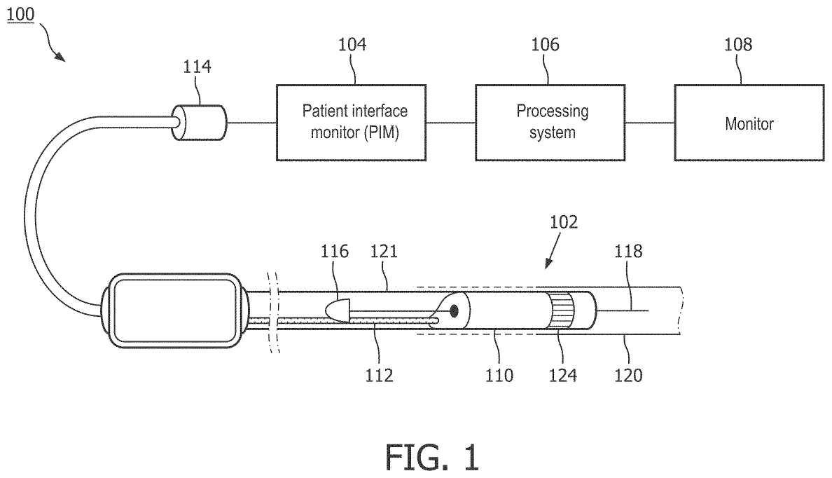

FIG. 1 is a diagrammatic schematic view of an ultrasound imaging system 100, according to aspects of the present disclosure. The ultrasound imaging system 100 can be an intraluminal imaging system. In some instances, the system 100 can be an intravascular ultrasound (IVUS) imaging system. The system 100 may include an intraluminal imaging device 102 such as a catheter, guide wire, or guide catheter, a patient interface module (PIM) 104, a processing system or console 106, and a monitor 108. The intraluminal imaging device 102 can be an ultrasound imaging device. In some instances, the device 102 can be IVUS imaging device, such as a solid-state IVUS device.

At a high level, the IVUS device 102 emits ultrasonic energy, or ultrasound signals, from a transducer array 124 included in scanner assembly 110 mounted near a distal end of the catheter device. The ultrasonic energy is reflected by tissue structures in the medium, such as a vessel 120, or another body lumen surrounding the scanner assembly 110, and the ultrasound echo signals are received by the transducer array 124. In that regard, the device 102 can be sized, shaped, or otherwise configured to be positioned within the body lumen of a patient. The PIM 104 transfers the received echo signals to the console or computer 106 where the ultrasound image (including the flow information) is reconstructed and displayed on the monitor 108. The console or computer 106 can include a processor and a memory. The computer or computing device 106 can be operable to facilitate the features of the IVUS imaging system 100 described herein. For example, the processor can execute computer readable instructions stored on the non-transitory tangible computer readable medium.

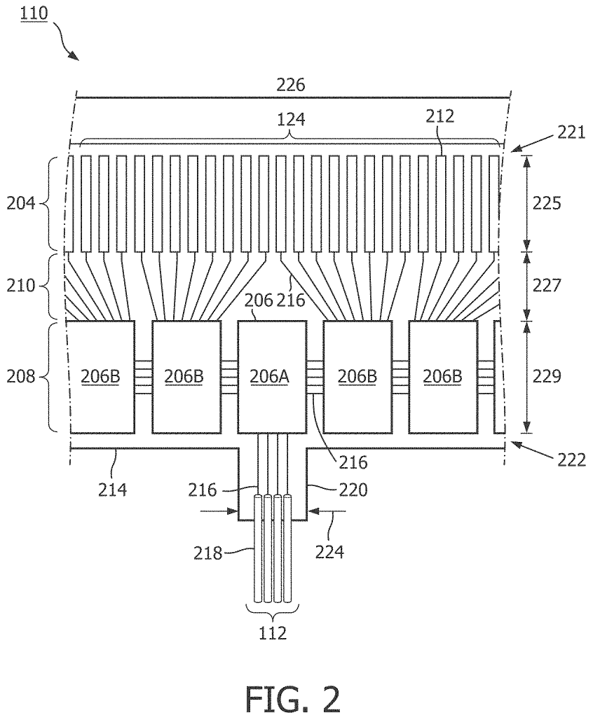

The PIM 104 facilitates communication of signals between the IVUS console 106 and the scanner assembly 110 included in the IVUS device 102. This communication includes the steps of: (1) providing commands to integrated circuit controller chip(s) 206A, 206B, illustrated in FIG. 2, included in the scanner assembly 110 to select the particular transducer array element(s), or acoustic element(s), to be used for transmit and receive, (2) providing the transmit trigger signals to the integrated circuit controller chip(s) 206A, 206B included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s)126 of the scanner assembly 110. In some embodiments, the PIM 104 performs preliminary processing of the echo data prior to relaying the data to the console 106. In examples of such embodiments, the PIM 104 performs amplification, filtering, and/or aggregating of the data. In an embodiment, the PIM 104 also supplies high- and low-voltage DC power to support operation of the device 102 including circuitry within the scanner assembly 110.

The IVUS console 106 receives the echo data from the scanner assembly 110 by way of the PIM 104 and processes the data to reconstruct an image of the tissue structures in the medium surrounding the scanner assembly 110. The console 106 outputs image data such that an image of the vessel 120, such as a cross-sectional image of the vessel 120, is displayed on the monitor 108. Vessel 120 may represent fluid filled or surrounded structures, both natural and man-made. The vessel 120 may be within a body of a patient. The vessel 120 may be a blood vessel, as an artery or a vein of a patient's vascular system, including cardiac vasculature, peripheral vasculature, neural vasculature, renal vasculature, and/or or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be used to examine man-made structures such as, but without limitation, heart valves, stents, shunts, filters and other devices.

In some embodiments, the IVUS device includes some features similar to traditional solid-state IVUS catheters, such as the EagleEye® catheter available from Volcano Corporation and those disclosed in U.S. Pat. No. 7,846,101 hereby incorporated by reference in its entirety. For example, the IVUS device 102 includes the scanner assembly 110 near a distal end of the device 102 and a transmission line bundle 112 extending along the longitudinal body of the device 102. The transmission line bundle or cable 112 can include a plurality of conductors, including one, two, three, four, five, six, seven, or more conductors 218 (FIG. 2). It is understood that any suitable gauge wire can be used for the conductors 218. In an embodiment, the cable 112 can include a four-conductor transmission line arrangement with, e.g., 41 AWG gauge wires. In an embodiment, the cable 112 can include a seven-conductor transmission line arrangement utilizing, e.g., 44 AWG gauge wires. In some embodiments, 43 AWG gauge wires can be used.

The transmission line bundle 112 terminates in a PIM connector 114 at a proximal end of the device 102. The PIM connector 114 electrically couples the transmission line bundle 112 to the PIM 104 and physically couples the IVUS device 102 to the PIM 104. In an embodiment, the IVUS device 102 further includes a guide wire exit port 116. Accordingly, in some instances the IVUS device is a rapid-exchange catheter. The guide wire exit port 116 allows a guide wire 118 to be inserted towards the distal end in order to direct the device 102 through the vessel 120.

In an embodiment, the image processing system 106 generates flow data by processing the echo signals from the IVUS device 102 into Doppler power or velocity information. The image processing system 106 may also generate B-mode data by applying envelope detection and logarithmic compression on the conditioned echo signals. The processing system 106 can further generate images in various views, such as 2D and/or 3D views, based on the flow data or the B-mode data. The processing system 106 can also perform various analyses and/or assessments. For example, the processing system 106 can apply virtual histology (VH) techniques, for example, to analyze or assess plaques within a vessel (e.g., the vessel 120). The images can be generated to display a reconstructed color-coded tissue map of plaque composition superimposed on a cross-sectional view of the vessel.

In an embodiment, the processing system 106 can apply a blood flow detection algorithm (e.g., ChromaFlo) to determine the movement of blood flow, for example, by acquiring image data of a target region (e.g., the vessel 120) repeatedly and determining the movement of the blood flow from the image data. The blood flow detection algorithm operates based on the principle that signals measured from vascular tissue are relatively static from acquisition to acquisition, whereas signals measured from blood flow vary at a characteristic rate corresponding to the flow rate. As such, the blood flow detection algorithm may determine movements of blood flow based on variations in signals measured from the target region between repeated acquisitions. To acquire the image data repeatedly, the processing system 106 may control to the device 102 to transmit repeated pulses on the same aperture.

While the present disclosure describes embodiments related to intravascular ultrasound (IVUS) imaging using an intravascular catheter or guidewire, it is understood that one or more aspects of the present disclosure can be implemented in any suitable ultrasound imaging system, including a synthetic aperture ultrasound imaging system, a phased array ultrasound imaging system, or any other array-based ultrasound imaging system. For example, aspects of the present disclosure can be implemented in intraluminal ultrasound imaging systems using an intracardiac (ICE) echocardiography catheter and/or a transesophageal echocardiography (TEE) probe, and/or external ultrasound imaging system using an ultrasound probe configured for imaging while positioned adjacent to and/or in contact with the patient's skin. The ultrasound imaging device can be a transthoracic echocardiography (TTE) imaging device in some embodiments.

An ultrasound transducer array of ultrasound imaging device includes an array of acoustic elements configured to emit ultrasound energy and receive echoes corresponding to the emitted ultrasound energy. In some instances, the array may include any number of ultrasound transducer elements. For example, the array can include between 2 acoustic elements and 10000 acoustic elements, including values such as 2 acoustic elements, 4 acoustic elements, acoustic elements, 64 acoustic elements, 128 acoustic elements, 500 acoustic elements, 812 acoustic elements, 3000 acoustic elements, 9000 acoustic elements, and/or other values both larger and smaller. In some instances, the transducer elements of the array may be arranged in any suitable configuration, such as a linear array, a planar array, a curved array, a curvilinear array, a circumferential array, an annular array, a phased array, a matrix array, a one-dimensional (1D) array, a 1.x dimensional array (e.g., a 1.5D array), or a two-dimensional (2D) array. The array of transducer elements (e.g., one or more rows, one or more columns, and/or one or more orientations) can be uniformly or independently controlled and activated. The array can be configured to obtain one-dimensional, two-dimensional, and/or three-dimensional images of patient anatomy.

The ultrasound transducer elements may comprise piezoelectric/piezoresistive elements, piezoelectric micromachined ultrasound transducer (PMUT) elements, capacitive micromachined ultrasound transducer (CMUT) elements, and/or any other suitable type of ultrasound transducer elements. The ultrasound transducer elements of the array are in communication with (e.g., electrically coupled to) electronic circuitry. For example, the electronic circuitry can include one or more transducer control logic dies. The electronic circuitry can include one or more integrated circuits (IC), such as application specific integrated circuits (ASICs). In some embodiments, one or more of the ICs can comprise a microbeamformer (μBF). In other embodiments, one or more of the ICs comprises a multiplexer circuit (MUX).

FIG. 2 is a diagrammatic top view of a portion of a flexible assembly 200, according to aspects of the present disclosure. The flexible assembly 200 includes a transducer array 124 formed in a transducer region 204 and transducer control logic dies 206 (including dies 206A and 206B) formed in a control region 208, with a transition region 210 disposed therebetween.

The transducer control logic dies 206 are mounted on a flexible substrate 214 into which the transducers 212 have been previously integrated. The flexible substrate 214 is shown in a flat configuration in FIG. 2. Though six control logic dies 206 are shown in FIG. 2, any number of control logic dies 206 may be used. For example, one, two, three, four, five, six, seven, eight, nine, ten, or more control logic dies 206 may be used.

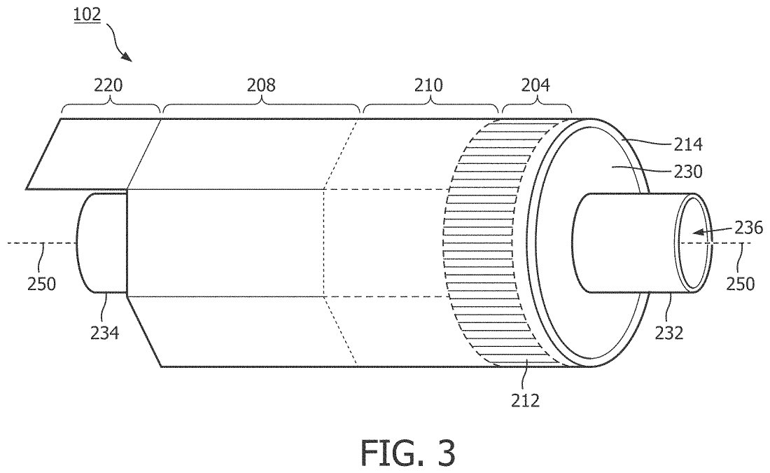

The flexible substrate 214, on which the transducer control logic dies 206 and the transducers 212 are mounted, provides structural support and interconnects for electrical coupling. The flexible substrate 214 may be constructed to include a film layer of a flexible polyimide material such as KAPTON™ (trademark of DuPont). Other suitable materials include polyester films, polyimide films, polyethylene napthalate films, or polyetherimide films, liquid crystal polymer, other flexible printed semiconductor substrates as well as products such as Upilex® (registered trademark of Ube Industries) and TEFLON® (registered trademark of E.I. du Pont). In the flat configuration illustrated in FIG. 2, the flexible substrate 214 has a generally rectangular shape. As shown and described herein, the flexible substrate 214 is configured to be wrapped around a support member 230 (FIG. 3) in some instances. Therefore, the thickness of the film layer of the flexible substrate 214 is generally related to the degree of curvature in the final assembled flexible assembly 110. In some embodiments, the film layer is between 5 μm and 100 μm, with some particular embodiments being between 5 μm and 25.1 μm, e.g., 6 μm.

The transducer control logic dies 206 is a non-limiting example of a control circuit. The transducer region 204 is disposed at a distal portion 221 of the flexible substrate 214. The control region 208 is disposed at a proximal portion 222 of the flexible substrate 214. The transition region 210 is disposed between the control region 208 and the transducer region 204. Dimensions of the transducer region 204, the control region 208, and the transition region 210 (e.g., lengths 225, 227, 229) can vary in different embodiments. In some embodiments, the lengths 225, 227, 229 can be substantially similar or, the length 227 of the transition region 210 may be less than lengths 225 and 229, the length 227 of the transition region 210 can be greater than lengths 225, 229 of the transducer region and controller region, respectively.

The control logic dies 206 are not necessarily homogenous. In some embodiments, a single controller is designated a master control logic die 206A and contains the communication interface for cable 142 which may serve as an electrical conductor, e.g., electrical conductor 112, between a processing system, e.g., processing system 106, and the flexible assembly 200. Accordingly, the master control circuit may include control logic that decodes control signals received over the cable 142, transmits control responses over the cable 142, amplifies echo signals, and/or transmits the echo signals over the cable 142. The remaining controllers are slave controllers 206B. The slave controllers 206B may include control logic that drives a transducer 212 to emit an ultrasonic signal and selects a transducer 212 to receive an echo. In the depicted embodiment, the master controller 206A does not directly control any transducers 212. In other embodiments, the master controller 206A drives the same number of transducers 212 as the slave controllers 206B or drives a reduced set of transducers 212 as compared to the slave controllers 206B. In an exemplary embodiment, a single master controller 206A and eight slave controllers 206B are provided with eight transducers assigned to each slave controller 206B.

To electrically interconnect the control logic dies 206 and the transducers 212, in an embodiment, the flexible substrate 214 includes conductive traces 216 formed in the film layer that carry signals between the control logic dies 206 and the transducers 212. In particular, the conductive traces 216 providing communication between the control logic dies 206 and the transducers 212 extend along the flexible substrate 214 within the transition region 210. In some instances, the conductive traces 216 can also facilitate electrical communication between the master controller 206A and the slave controllers 206B. The conductive traces 216 can also provide a set of conductive pads that contact the conductors 218 of cable 142 when the conductors 218 of the cable 142 are mechanically and electrically coupled to the flexible substrate 214. Suitable materials for the conductive traces 216 include copper, gold, aluminum, silver, tantalum, nickel, and tin, and may be deposited on the flexible substrate 214 by processes such as sputtering, plating, and etching. In an embodiment, the flexible substrate 214 includes a chromium adhesion layer. The width and thickness of the conductive traces 216 are selected to provide proper conductivity and resilience when the flexible substrate 214 is rolled. In that regard, an exemplary range for the thickness of a conductive trace 216 and/or conductive pad is between 1-5 μm. For example, in an embodiment, 5 μm conductive traces 216 are separated by 5 μm of space. The width of a conductive trace 216 on the flexible substrate may be further determined by the width of the conductor 218 to be coupled to the trace/pad.

The flexible substrate 214 can include a conductor interface 220 in some embodiments. The conductor interface 220 can be a location of the flexible substrate 214 where the conductors 218 of the cable 142 are coupled to the flexible substrate 214. For example, the bare conductors of the cable 142 are electrically coupled to the flexible substrate 214 at the conductor interface 220. The conductor interface 220 can be tab extending from the main body of flexible substrate 214. In that regard, the main body of the flexible substrate 214 can refer collectively to the transducer region 204, controller region 208, and the transition region 210. In the illustrated embodiment, the conductor interface 220 extends from the proximal portion 222 of the flexible substrate 214. In other embodiments, the conductor interface 220 is positioned at other parts of the flexible substrate 214, such as the distal portion 221, or the flexible substrate 214 may lack the conductor interface 220. A value of a dimension of the tab or conductor interface 220, such as a width 224, can be less than the value of a dimension of the main body of the flexible substrate 214, such as a width 226. In some embodiments, the substrate forming the conductor interface 220 is made of the same material(s) and/or is similarly flexible as the flexible substrate 214. In other embodiments, the conductor interface 220 is made of different materials and/or is comparatively more rigid than the flexible substrate 214. For example, the conductor interface 220 can be made of a plastic, thermoplastic, polymer, hard polymer, etc., including polyoxymethylene (e.g., DELRIN®), polyether ether ketone (PEEK), nylon, Liquid Crystal Polymer (LCP), and/or other suitable materials.

FIG. 3 illustrates a perspective view of the device 102 with the scanner assembly 110 in a rolled configuration. In some instances, the assembly 110 is transitioned from a flat configuration (FIG. 2) to a rolled or more cylindrical configuration (FIG. 3). For example, in some embodiments, techniques are utilized as disclosed in one or more of U.S. Pat. No. 6,776,763, titled “ULTRASONIC TRANSDUCER ARRAY AND METHOD OF MANUFACTURING THE SAME” and U.S. Pat. No. 7,226,417, titled “HIGH RESOLUTION INTRAVASCULAR ULTRASOUND SENSING ASSEMBLY HAVING A FLEXIBLE SUBSTRATE,” each of which is hereby incorporated by reference in its entirety.

In some embodiments, the transducer elements 212 and/or the controllers 206 can be positioned in in an annular configuration, such as a circular configuration or in a polygon configuration, around a longitudinal axis 250 of a support member 230. It will be understood that the longitudinal axis 250 of the support member 230 may also be referred to as the longitudinal axis of the scanner assembly 110, the flexible elongate member 121, and/or the device 102. For example, a cross-sectional profile of the imaging assembly 110 at the transducer elements 212 and/or the controllers 206 can be a circle or a polygon. Any suitable annular polygon shape can be implemented, such as a based on the number of controllers/transducers, flexibility of the controllers/transducers, etc., including a pentagon, hexagon, heptagon, octagon, nonagon, decagon, etc. In some examples, the plurality of transducer controllers 206 may be used for controlling the plurality of ultrasound transducer elements 212 to obtain imaging data associated with the vessel 120.

The support member 230 can be referenced as a unibody in some instances. The support member 230 can be composed of a metallic material, such as stainless steel, or non-metallic material, such as a plastic or polymer as described in U.S. Provisional Application No. 61/985,220, “Pre-Doped Solid Substrate for Intravascular Devices,” filed Apr. 28, 2014, ('220 Application) the entirety of which is hereby incorporated by reference herein. The support member 230 can be a ferrule having a distal flange or portion 232 and a proximal flange or portion 234. The support member 230 can be tubular in shape and define a lumen 236 extending longitudinally therethrough. The lumen 236 can be sized and shaped to receive the guide wire 118. The support member 230 can be manufactured using any suitable process. For example, the support member 230 can be machined and/or electrochemically machined or laser milled, such as by removing material from a blank to shape the support member 230, or molded, such as by an injection molding process.

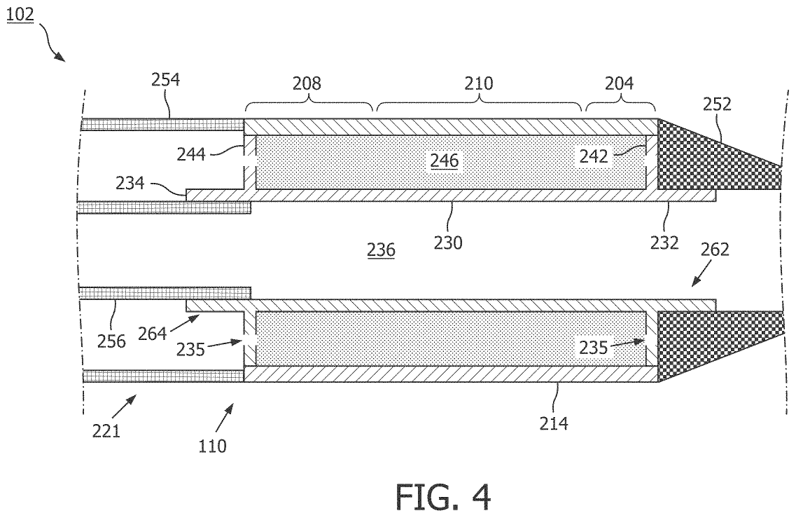

Referring now to FIG. 4, shown there is a diagrammatic cross-sectional side view of a distal portion of the intraluminal imaging device 102, including the flexible substrate 214 and the support member 230, according to aspects of the present disclosure. The support member 230 can be referenced as a unibody in some instances. The support member 230 can be composed of a metallic material, such as stainless steel, or non-metallic material, such as a plastic or polymer as described in U.S. Provisional Application No. 61/985,220, “Pre-Doped Solid Substrate for Intravascular Devices,” filed Apr. 28, 2014, the entirety of which is hereby incorporated by reference herein. The support member 230 can be ferrule having a distal portion 262 and a proximal portion 264. The support member 230 can define a lumen 236 extending along the longitudinal axis LA. The lumen 236 is in communication with the entry/exit port 116 and is sized and shaped to receive the guide wire 118 (FIG. 1). The support member 230 can be manufactured according to any suitable process. For example, the support member 230 can be machined and/or electrochemically machined or laser milled, such as by removing material from a blank to shape the support member 230, or molded, such as by an injection molding process. In some embodiments, the support member 230 may be integrally formed as a unitary structure, while in other embodiments the support member 230 may be formed of different components, such as a ferrule and stands 242, 244, that are fixedly coupled to one another. In some cases, the support member 230 and/or one or more components thereof may be completely integrated with inner member 256. In some cases, the inner member 256 and the support member 230 may be joined as one, e.g., in the case of a polymer support member.

Stands 242, 244 that extend vertically are provided at the distal and proximal portions 262, 264, respectively, of the support member 230. The stands 242, 244 elevate and support the distal and proximal portions of the flexible substrate 214. In that regard, portions of the flexible substrate 214, such as the transducer portion 204 (or transducer region 204), can be spaced from a central body portion of the support member 230 extending between the stands 242, 244. The stands 242, 244 can have the same outer diameter or different outer diameters. For example, the distal stand 242 can have a larger or smaller outer diameter than the proximal stand 244 and can also have special features for rotational alignment as well as control chip placement and connection. To improve acoustic performance, any cavities between the flexible substrate 214 and the surface of the support member 230 are filled with a backing material 246. The liquid backing material 246 can be introduced between the flexible substrate 214 and the support member 230 via passageways 235 in the stands 242, 244. In some embodiments, suction can be applied via the passageways 235 of one of the stands 242, 244, while the liquid backing material 246 is fed between the flexible substrate 214 and the support member 230 via the passageways 235 of the other of the stands 242, 244. The backing material can be cured to allow it to solidify and set. In various embodiments, the support member 230 includes more than two stands 242, 244, only one of the stands 242, 244, or neither of the stands. In that regard the support member 230 can have an increased diameter distal portion 262 and/or increased diameter proximal portion 264 that is sized and shaped to elevate and support the distal and/or proximal portions of the flexible substrate 214.

The support member 230 can be substantially cylindrical in some embodiments. Other shapes of the support member 230 are also contemplated including geometrical, non-geometrical, symmetrical, non-symmetrical, cross-sectional profiles. As the term is used herein, the shape of the support member 230 may reference a cross-sectional profile of the support member 230. Different portions the support member 230 can be variously shaped in other embodiments. For example, the proximal portion 264 can have a larger outer diameter than the outer diameters of the distal portion 262 or a central portion extending between the distal and proximal portions 262, 264. In some embodiments, an inner diameter of the support member 230 (e.g., the diameter of the lumen 236) can correspondingly increase or decrease as the outer diameter changes. In other embodiments, the inner diameter of the support member 230 remains the same despite variations in the outer diameter.

A proximal inner member 256 and a proximal outer member 254 are coupled to the proximal portion 264 of the support member 230. The proximal inner member 256 and/or the proximal outer member 254 can comprise a flexible elongate member. The proximal inner member 256 can be received within a proximal flange 234. The proximal outer member 254 abuts and is in contact with the flexible substrate 214. A distal member 252 is coupled to the distal portion 262 of the support member 230. For example, the distal member 252 is positioned around the distal flange 232. The distal member 252 can abut and be in contact with the flexible substrate 214 and the stand 242. The distal member 252 can be the distal-most component of the intraluminal imaging device 102.

One or more adhesives can be disposed between various components at the distal portion of the intraluminal imaging device 102. For example, one or more of the flexible substrate 214, the support member 230, the distal member 252, the proximal inner member 256, and/or the proximal outer member 254 can be coupled to one another via an adhesive.

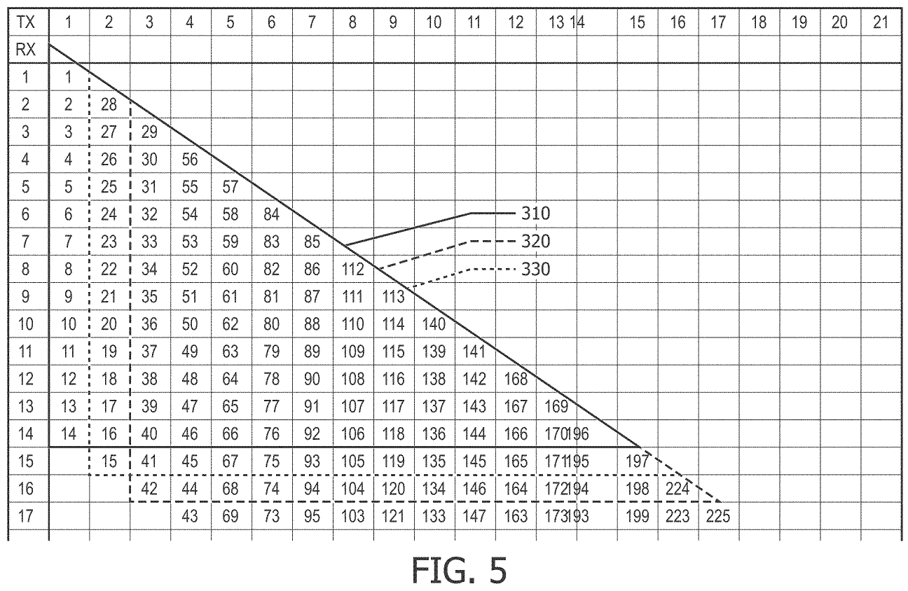

The assembly 110 shown in FIG. 2 can be activated according to a pulse sequence or scan sequence to form coherent beams of ultrasound energy to form an image. In that regard, FIG. 5 is a diagrammatic graphical view showing an ultrasound pulse sequence of a solid-state IVUS device. The pulse sequence 300 comprises a contiguous “zig-zag” pattern or arrangement of transmit-receive pairs, which can alternatively be described as transmit-receive events. Each transmit-receive pair is represented by an index, or number, corresponding to a sequential time at which the corresponding transmit-receive pair is activated to obtain ultrasound imaging data. In that regard, each transmit-receive index is an integer representing its relative temporal position in the sequence 300. In the embodiment of FIG. 5, each transmit-receive index corresponds to a single transmit-receive pair. Each transmit-receive pair is defined by a transmit element index, shown on the x-axis, and a receive element index, shown on the y-axis. Each transmit element index and receive element index corresponds to an ultrasound element of an array of ultrasound transducer elements. In the embodiment shown in FIG. 5, the array comprises 64 ultrasound transducer elements.

For example, the transmit-receive pair associated with transmit-receive index “1” is defined by transmit element index number 1 and receive element index 1. In some embodiments, the transmit element index and receive element index correspond to the same ultrasound transducer element. In other embodiments, the transmit element index and receive element index correspond to different ultrasound transducer elements. For example, the transmit-receive pair numbered “2,” which is shown directly below transmit-receive pair 1, is defined by transmit element index 1 and receive element index 2. That is, the ultrasound imaging data associated with transmit-receive pair 2 is obtained by activating transmit element index 1 to transmit ultrasound energy into the patient volume, and then activating receive element index 2 to receive ultrasound echoes from the patient volume. In FIG. 5, 294 transmit-receive pairs of an ultrasound pulse sequence are shown. Each transmit-receive pair is activated sequentially according to its transmit-receive index.

In the sequence 300, the ultrasound transducer element associated with transmit index 1 transmits 14 consecutive times, while the elements associated with receive indices 1 through 14 are sequentially activated to receive the corresponding echoes. Next, the element associated with transmit index 2 transmits 14 consecutive times, while the elements associated with receive indices 15 through 2 (stepping backward) are sequentially activated to receive the corresponding echoes. This sequence continues in a zig-zag pattern around the array of ultrasound transducer elements. Each transmit-receive pair is associated with one or more apertures 310, 320, 330. For example, a first aperture 310 comprises transmit-receive pairs spanning from index 1 to index 196, a second aperture 320 comprises transmit-receive pairs spanning from index 15 to index 197, and a third aperture 330 comprises transmit-receive pairs spanning from index 29 to index 224. The transmit-receive pairs in each aperture are combined to form an A-line for a B-mode image. Thus, the transmit-receive pairs contained within the first aperture 310 are combined to form a first A-line, the transmit-receive pairs contained within the second aperture 320 are combined to form a second A-line, the transmit-receive pairs contained within the third aperture are combined to form a third A-line, and so on. The A-line formed by the first aperture 310 will be centered between transmit and receive element indices 7 and 8, the A-line formed by the second aperture 320 will be centered between transmit and receive element indices numbered 8 and 9, the A-line formed by the third aperture 330 will be centered between transmit and receive element indices numbered 9 and 10, and so on. Several apertures are used to form A-lines, which are combined and arranged to form a B-mode image.

It will be understood that the scan sequence shown in FIG. 5 is exemplary and that other scanning sequences can be used besides that sequence shown in FIG. 5. For example the present disclosure contemplates scan sequences using different patterns of transmit-receive pairs, different aperture sizes, and different combinations of transmit and receive pairs.

Grating lobe artifacts can appear in an image due to one or more off-axis objects reflecting an unfocused portion (e.g., a grating lobe) of an ultrasound pulse back to the acoustic elements of the array. Ultrasound transducer arrays that do not satisfy the Nyquist criteria may be particularly susceptible to producing grating lobe artifacts. Grating lobe artifacts can appear in B-mode ultrasound images as blurry duplicates of the off-axis target. Grating lobe artifacts add unwanted image clutter that complicates the image analysis process and makes it difficult for the physician or ultrasound technician to interpret ultrasound images, such as the tissue structure of a blood vessel.

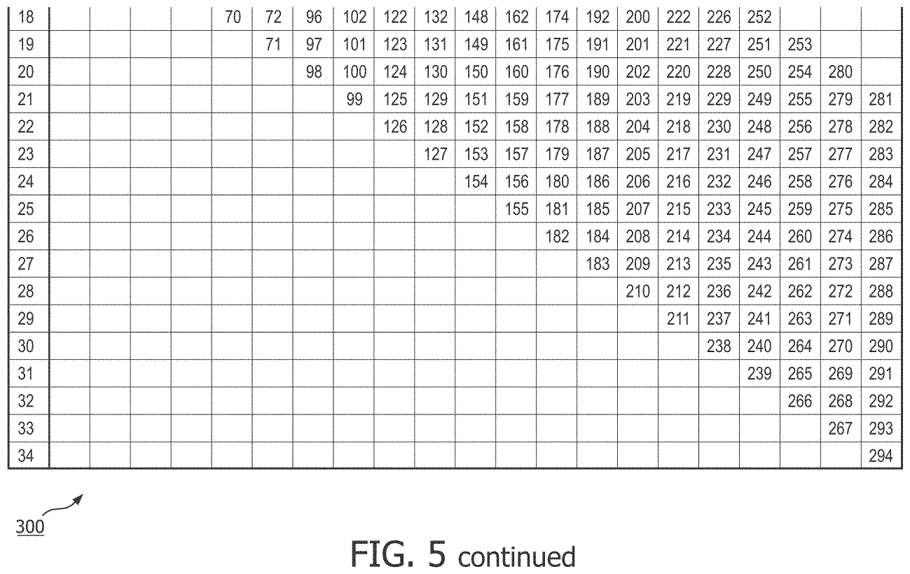

FIGS. 6A and 6B depict a portion of an array 400 of acoustic elements receiving ultrasound signals reflected by a target object 410 at different positions relative to a center axis 416 of an aperture. The array 400 is an IVUS imaging array disposed in a circular pattern with a radius of R around a circumference of the imaging assembly. However, any array of acoustic elements is contemplated, such as a linear array, a curvilinear array, arrays for external ultrasound, two-dimensional arrays, etc. As shown in FIGS. 6A and 6B, a first portion 422 of the acoustic elements of the array are grouped together and activated as an aperture, while other acoustic elements 424, which are not part of the aperture, are not activated. The acoustic elements 422 of the aperture are activated according to a scan sequence to transmit ultrasound energy and receive echoes associated with the transmitted signals. In some embodiments, the scan sequence is performed by activating a single acoustic element at a time to transmit ultrasound energy and/or receive echoes corresponding to the transmitted ultrasound energy. The position or index of each element in the aperture can be described by the angle ft between the line perpendicular to the center of the active aperture and the line perpendicular to the element.

In FIG. 6A, a target object 410 is shown at a first, on-axis position reflecting ultrasound energy to the elements of the array 400. The first portion 422 of the elements of the array are activated as an aperture, which includes the shaded elements. A second portion 424 of the elements of the array are not activated and not included in the aperture. The aperture includes a first element 426 and a second element 428. The target object 410 reflects a first portion 412 of ultrasonic energy toward the first element 426, and a second portion 414 of ultrasonic energy toward the second element 428. Each element of the array 400 can receive ultrasonic energy from a range of angles of acceptance. The angle of acceptance θ is the angle between the radial center line 418 of the second element 428 and the direction of the received second portion 414 of ultrasonic energy. In that regard, because the first portion 412 of energy is aligned with the radial center line of the first element 426, and therefore aligned with the center of the aperture (i.e. β=0°), the angle of acceptance of the first portion 412 of ultrasonic energy by the first element 426 is 0°. By contrast, as the second element 428 is off-center from the aperture (i.e. β≠0°), the angle of acceptance θ of the second portion 414 of ultrasonic energy on the second element 428 is significantly larger, for example, approximately 55°. However, because the first and second portions 412, 414 of ultrasonic energy are reflected by the target object 410 when it is aligned with the center axis 416 of the aperture, both the first and second portions 412, 414 of ultrasonic energy are considered to be on-axis contributions to the signal received by the aperture.

In FIG. 6B, the target object 410 is shown at a second, off-axis position reflecting ultrasound energy to the elements of the array 400. The target object 410 reflects a first portion 412′ of energy to the first element 426, positioned at the center of the aperture 422, and a second portion 414′ of energy to the second element 428. Although the angle of acceptance θ′ between the center axis 418 of the second element 428 is closer to 0° than the angle θ of acceptance shown in FIG. 6A, the angle of arrival φ, which measured between the center axis 416 of the aperture (i.e. the first element 426) and the first portion of energy 412′, is significantly greater than 0°. In other words, the energy reflected by the target object 410 when it is in the second position is off-axis (i.e. φ≠0). By contrast, when the target object 410 is in the first position, energy reflected by the target object toward the aperture is on-axis (i.e. φ=0).

As mentioned above, grating lobe artifacts appear because a portion of ultrasonic energy is reflected from off-axis objects to elements in an aperture. In other words, grating lobe artifacts result when acoustic elements of a spatially-undersampled array receive ultrasonic energy at relatively large angles of acceptance. The present disclosure describes systems and methods for reducing grating lobe artifacts by taking into account the directional amplitude response and directivity of the array to reduce the influence of ultrasound signals received by off-axis acoustic elements. In some aspects, embodiments of the present disclosure involve using a known directivity and amplitude response of the acoustic elements of an aperture to isolate on-axis signals and disregard off-axis signals that cause grating lobe artifacts.

Each individual element of an array can receive ultrasound energy from a wide range of acceptance angles. However, the amplitude of a signal generated by an element changes depending on the angle of acceptance. This characteristic of acoustic elements in an array is referred to as directivity. The directivity of the acoustic elements in the array can be described as the directional sensitivity of the acoustic elements to signals received at different angles. The directivity of the elements is manifested in the amplitude response of an aperture, and can be characterized by the mathematical relationship:

$$f ( θ ) = \frac{sin ( π d / λ sin θ )}{π d / λ sin θ} cos θ$$

where d is the element width, A is the wavelength of the received ultrasonic energy, and θ is the acceptance angle between the target object (e.g, target object 410) or source of the signal and the element. For a circular array, such as an array of an IVUS imaging catheter, the acceptance angle θ can be determined using the mathematical relationship:

$$θ = β + {tan}^{- 1} ( \frac{R sin β}{R ( 1 - cos β ) + r} )$$

Where R is the radius of curvature of the array, and r is the range or distance between the target object and the center of the active aperture. It will be understood that θ can be determined by other relationships when the array is not circular. It will be further understood that the relationship for directivity shown above may be approximate and based upon certain boundary conditions. In practice, acoustic elements may have a more rapid “fall off” as a function of angle than expressed by the relationship above. For example, Selfridge, et al., “A theory for the radiation patter of a narrow-strip acoustic transducer,” Appl. Phys. Lett. 37(a), Jul. 1, 1980, which is hereby incorporated by reference in its entirety, provides further information and relationships for characterizing the directivity of an array of acoustic elements.

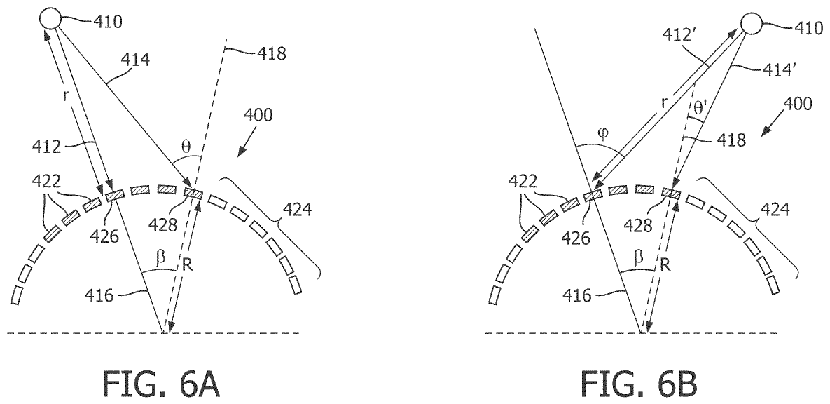

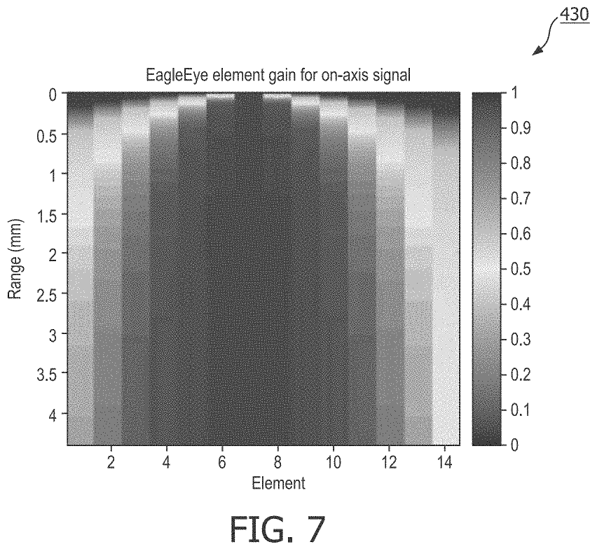

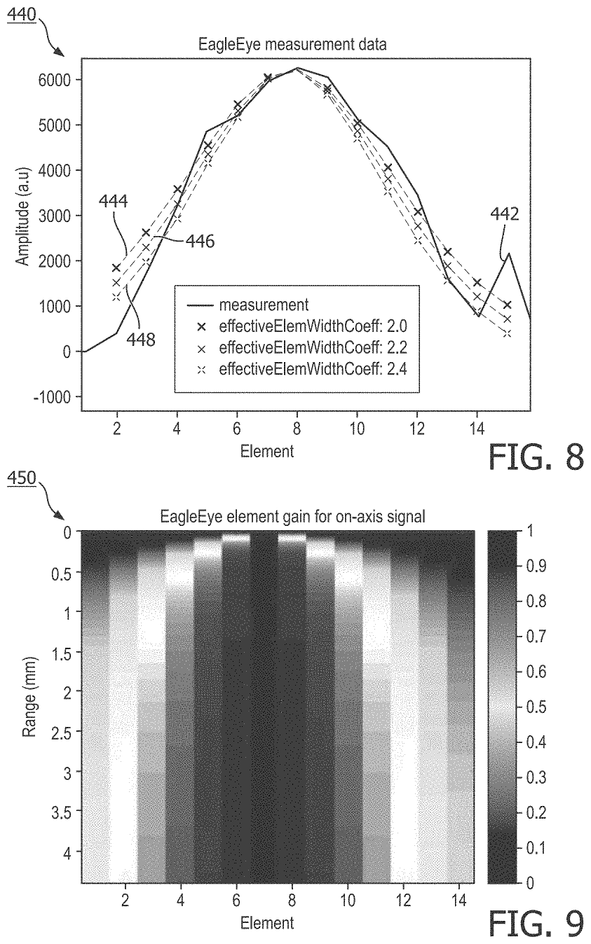

By choosing several different values for an angle of arrival, which corresponds to different target positions relative to an aperture, a directional amplitude response can be obtained for each element in an aperture. In that regard, FIG. 7 is a graph 430 that shows the amplitude response across an aperture for an on-axis signal. As shown, the on-axis signal is centered on element 7 of a 14-element aperture. The amplitude response depends on both the element index (which relates to the angle of arrival φ) and the range or depth. For the on-axis signal, the amplitude response is highest at the center of the aperture and decreases toward the edges of the aperture. It will be understood that the amplitude response shown in the graph 430 of FIG. 7 can be determined using only geometric parameters (e.g., radius, element spacing) of the array, independent of image data obtained by the array. While this theoretically-derived amplitude response can be used to approximate the amplitude response of an array of acoustic elements, empirical data can be used to compensate for crosstalk between elements of the array, which causes an artificially wider element width effect, as well as other real-world effects that cause the amplitude response to deviate from the theoretical approximation shown in FIG. 7. This effect can be shown in the graph 440 of FIG. 8, which shows a plot 442 of a measured amplitude response of an aperture and three fit curves 444, 446, 448 having different element width coefficients to determine a best-fitting element width coefficient. By modifying the amplitude response shown in FIG. 7 with the coefficients determined by experimental data shown in FIG. 8, a corrected amplitude response graph 450 is obtained and shown in FIG. 9. The corrected amplitude response graph 450 is more tightly centered around the center element and decreases more abruptly toward the edges of the aperture.

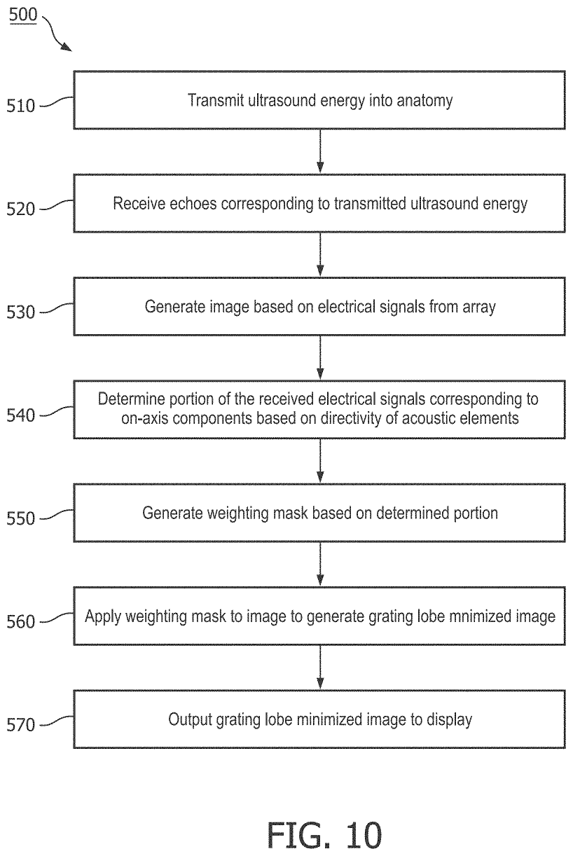

Because grating lobe artifacts are the result of ultrasonic signals reflected by objects that are off-axis from an aperture, the known directivity of the elements and the amplitude response of the aperture can be used to reduce, minimize, or eliminate the presence of grating lobe artifacts, even for a spatially-undersampled array. For example, the weight of signals from off-axis targets can be adjusted based on the angle of arrival of the signals and the directivity of the acoustic elements in the array. In that regard, FIG. 10 illustrates a method 500 for reducing or minimizing grating lobe artifacts in an ultrasound image.

In step 510, an array of acoustic elements transmits ultrasonic energy into an anatomy of a patient. The array of acoustic elements may be controlled by a controller or processor to transmit the ultrasonic energy according to a scan sequence comprising a plurality of apertures and subapertures. In some embodiments, the scan sequence may comprise activating a single acoustic element at a given time to transmit and/or receive acoustic energy. In other embodiments, the scan sequence may comprise simultaneously activating a plurality of acoustic elements to transmit and/or receive acoustic energy. In some embodiments, the processor may comprise a single processing component, or several processing components. In some embodiments, one or more of the processing components may be coupled to the array of acoustic elements. For example, the processor may comprise the ASIC controller 206 of the scanner assembly 110 shown in FIG. 2. In the same or other embodiments, one or more of the processing components may be coupled to the array of acoustic elements by an electrical cable. For example, one or more of the processing components may be disposed in a central console and may be configured to send and receive electrical signals to and from the array via an electrical connector.

In step 520, the array receives ultrasonic echoes corresponding to the transmitted ultrasonic energy. The array may be controlled by the processor to activate one or more apertures to receive the echoes. The apertures can be characterized by a plurality of transmit-receive pairs spanning one or more acoustic elements of the array. The array generates electrical signals or image data in response to receiving the echoes and transmits the electrical signals to the processor over a plurality of communication channels. In step 530, the processor generates an original image using the received image data. In some embodiments, generating the image includes aligning the image data from the plurality of channels using delay-and-sum beamforming. Particularly if the array is spatially undersampled, the image generated in step 530 may include grating lobe artifacts. Steps 540-560 are directed to generating a grating lobe minimized image in which grating lobe artifacts present in the original image generated in step 530 are reduced, minimized, or eliminated.

In step 540, the processor determines a fraction or portion associated with the on-axis components or contributions of the image data received across the plurality of channels. For example, the fraction or portion associated with the on-axis components may be described as an estimated ratio of a detected signal that arises from the on-axis direction to the total detected signal at any given location. The fraction or portion is determined by taking into account the directivity and directional amplitude response of the elements of the array. In an N channel system, and at any given depth, the amplitude (or envelope) of the time-aligned measurement data across an N element aperture can be described by the equation:

S=[s₁ s₂ S₃ . . . s_{N}]^{T}

The raw channel data (i.e. real-only signal) is Hilbert transformed, and then the absolute value of the Hilbert-transformed data is taken to obtain the amplitude or envelope needed to construct the vector S. In some embodiments, each element in the vector S can be averaged over the range dimension to improve the robustness of the algorithm. As described above, the amplitude response of the N-element aperture can be determined for several different angles of arrival. A matrix G can be assembled that includes amplitude responses of the elements of the aperture based on M different angles of arrival φ. Thus, the matrix G can be of the form:

$$G = [ \begin{matrix}{g}_{1 , {ϕ}_{1}} & {g}_{1 , {ϕ}_{2}} & \text{...} & {g}_{1 , {ϕ}_{M}} \\ {g}_{2 , {ϕ}_{1}} & {g}_{2 , {ϕ}_{2}} & \text{...} & {g}_{2 , {ϕ}_{M}} \\ {g}_{3 , {ϕ}_{1}} & {g}_{3 , {ϕ}_{2}} & \text{...} & {g}_{3 , {ϕ}_{M}} \\ ⋮ & ⋮ & ⋱ & ⋮ \\ {g}_{N , {ϕ}_{1}} & {g}_{N , {ϕ}_{2}} & \text{...} & {g}_{N , {ϕ}_{M}}\end{matrix} ] ,$$

where each column vector in G corresponds to an amplitude response basis vector specified by the angle of arrival φ. As described above, the angle of arrival φis similar to acceptance angle θ, in that the angle of arrival φ is also related to the direction from which ultrasound energy is received by the elements of the array. However, whereas the acceptance angle θ is measured relative to each acoustic element of the array, the angle of arrival φ is measured relative to the center of the aperture.

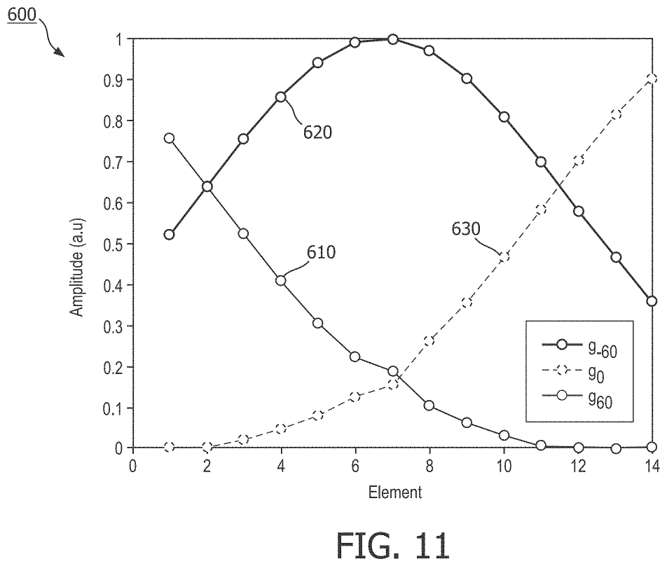

FIG. 11 is a graph that shows three different amplitude response basis vectors obtained from matrix G for a 14-element aperture, at a particular depth, corresponding to three different angles of arrival (φ=−60°, φ=0°, φ=60°). As shown in FIG. 11, the first plot corresponding to an on-axis signal (φ=0°) has a maximum centered about element 7, which is the center of the aperture. By contrast, the amplitudes of the second and third plots, which correspond to angles of arrival of −60° and 60°, respectively, are highest at the edges of the aperture, and are significantly lower than the on-axis signal near the center of the aperture. These directivity-based differences in the amplitude response of the aperture can be exploited to distinguish or separate portions of a signal depending on the angle at which the signal reaches the aperture. In particular, the on-axis portion (φ=0°) of the signal can be isolated to generate a weighting mask that reduces or minimizes the presence of grating lobe artifacts, as explained further below.

Referring again to step 540 of the method 500, whereas the elements of vector S relate to the portion of the signal amplitude generated by different elements of the aperture, it may be desirable to calculate or determine a vector whose components relate to the portion of the signal amplitude contributed by different angles of arrival. In that regard, determining the fraction or portion of on-axis and off-axis signal components of S can include performing, by the processor, a decomposition of the vector S into the amplitude basis vectors of G corresponding to different angles of arrival. For example, the vector S can be decomposed into M amplitude basis vectors in matrix G using the following relationship:

S=GW, where

W=[ω_{φ}_{₁} ω_{φ}_{₂} ω_{φ}_{₃} . . . ω_{φ}_{_{M}}]^{T},

and where W is the weight vector whose elements correspond to signal contributions from the M different angles of arrival. Accordingly, by decomposing the vector S to obtain the elements of the weight vector W, the portion of signal or amplitude S corresponding to a variety of given angles of arrival φ can be determined. Particularly, the portion of signal that is on-axis (φ=0°) can be determined or isolated, while the off-axis portions of the signal (φ≠0°) can be ignored and/or reduced. By ignoring/reducing the effects of off-axis signals, grating lobe artifacts can be reduced, minimized, or eliminated.

In an exemplary embodiment, the processor uses a least squares solution to decompose the vector S and obtain the elements of the weight vector W. Accordingly, W can be derived by minimizing the squared error between vector S and the weighted sum of basis vectors GW according to the following relationship:

e=Σ(S-GW)²

The least squares solution can then be obtained by the following equation:

$$w = \frac{{G}^{T} S}{{G}^{T} G}$$

In some embodiments, a regularization term for the squared error e could be used to find the solution for W to improve the performance of the algorithm.

The determined weight vector W can then be normalized by the processor according to the equation:

$${W}^{′} = \frac{W}{{\sum }_{i = 1}^{M} {ω}_{{ϕ}_{i}}}$$

$$where \text{:} {W}^{′} = {[ {ω}_{{ϕ}_{1}}^{′} {ω}_{{ϕ}_{2}}^{′} {ω}_{{ϕ}_{3}}^{′} \text{...} {ω}_{{ϕ}_{M}}^{′} ]}^{T}$$

and where each element ω′_{φ} corresponds to the normalized portion or fraction of components of the received image data from the N channels corresponding to angle of arrival gyp.

In step 550, once the elements of W′ are determined, the processor generates or computes a weighting mask m(p, ϕ) based on the following relationship:

m(ρ, ϕ)=ω′_{φ=0°}(ρ, ϕ),

where ω′_{φ=0°} corresponds to the portion or fraction of on-axis (i.e., φ=0°) signals, and ρ and ϕ correspond to the depth/scan line coordinates in the image. Thus, the weighting mask m(ρ, ϕ) is generated by isolating the normalized portion or fraction of signal that are aligned with the center or axis of the aperture. In step 560, a grating lobe minimized image I(ρ, ϕ) can be generated by applying the weighting mask m(ρ, ϕ) to the original image I_{original} (ρ, ϕ), which may include significant grating lobe artifacts. For example, the weighting mask m(ρ, ϕ) could be applied to the original image I_{original} (ρ, ϕ) by multiplying the weighting mask m(ρ, ϕ) with the original image I_{original} (ρ, ϕ), as shown by the following relationship:

I(ρ, ϕ)=I_{original} (ρ, ϕ) * m(ρ, ϕ)

In step 570, the processor outputs the grating lobe minimized image I(ρ, ϕ) to a display.

It will be understood that the present disclosure contemplates other methods to obtain the weight vector W and/or apply the weighting mask m(ρ, ϕ) to the original image I_{original}(ρ, ϕ). For example, in one embodiment, a linear least squares approach is used in order to obtain the weight vector W. Any suitable approach can be used to decompose S into G to obtain W. It will also be understood that, while the particular mathematical relationships described above are performed in linear domain, in some embodiments, the weighting mask m(ρ, ϕ) could be determined in log domain. Further, in some embodiments, the weighting mask m(ρ, ϕ) can be applied to the original image I_{original}(ρ, ϕ) in ways other than multiplication, such as by converting the original image I_{orginal}(ρ, ϕ) to log domain and subtracting a log domain weighting mask m from the original image I_{original}(ρ, ϕ). Any suitable method can be used to apply the weighting mask m(ρ, ϕ) to the original image I_{original}(ρ, ϕ).

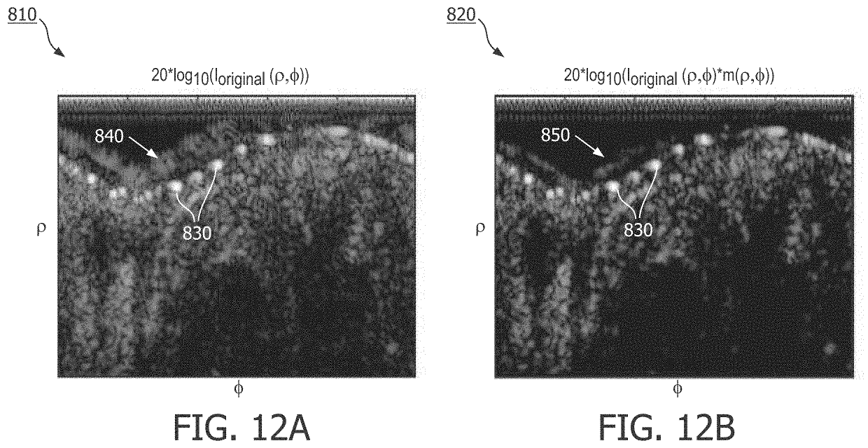

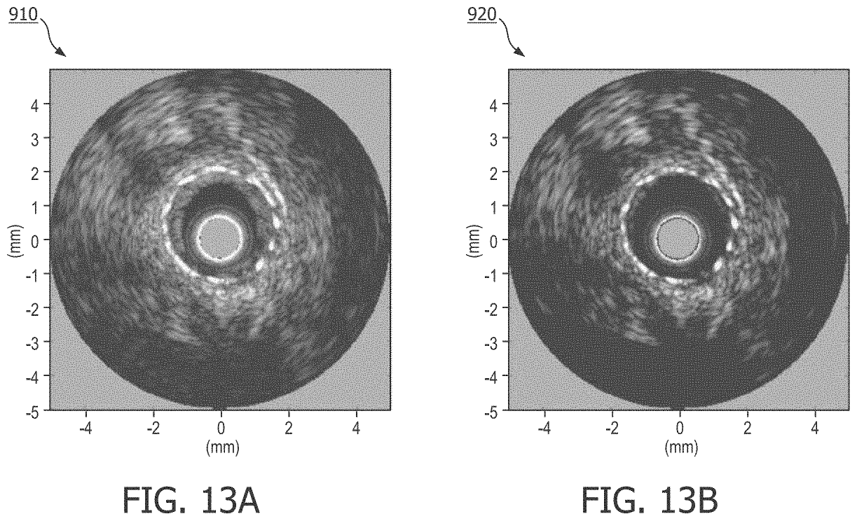

FIGS. 12A and 12B show an original IVUS image 810 having significant grating lobe artifacts, and a grating lobe minimized or reduced IVUS image 820, respectively, in ρ/ϕ domain before scan conversion. The original IVUS image 810 represents a cross-sectional view of a blood vessel including a stent having stent struts 830. The original image 810 also shows grating lobe artifacts 840, which may be caused, in part, by off-axis reflections from the stent struts 830. FIG. 12B shows a grating lobe minimized image 820 generated from the original image 810 using the method 500 described above. In contrast to the original image 810, the area 850 of the grating lobe minimized image 820, which corresponds to the area in which the grating lobe artifacts 840 are located in the original image 810, includes significantly reduced grating lobe artifacts. However, the grating lobe minimized image 820 includes all or a substantial portion of the remaining image features of the original image 810, such as the stent struts 830, vessel structure, tissue speckle pattern, etc. FIGS. 13A and 13B depict the original IVUS 910 image with grating lobe artifacts and the grating lobe minimized IVUS image 920 with reduced grating lobe artifacts after scan conversion to form circular cross-sectional views of the blood vessel and stent structure. In some embodiments, the scan-converted grating lobe minimized image 920 is the image that is output to the display. In some embodiments, the pre-scan-converted grating lobe minimized image 820 is the image that is output to the display. In other embodiments, both the scan-converted and the pre-scan-converted grating lobe minimized images 820, 920 are output to the display.

It will be understood that one or more of the steps of the method 500, such as controlling the array to transmit and receive ultrasound energy, generating the image, determining the fraction or portion, generating the grating lobe minimized image, and outputting the grating lobe minimized image to the display, can be performed by one or more components of an ultrasound imaging system, such as the processor, a multiplexer, a beamformer, a signal processing unit, an image processing unit, or any other suitable component of the system. For example, activating a scan sequence may be carried out by a processor in communication with a multiplexer configured to select or activate one or more elements of an ultrasound transducer array. In some embodiments, generating the ultrasound images may include beamforming incoming signals from the ultrasound imaging device and processing the beamformed signals by an image processor. The processing components of the system can be integrated within the ultrasound imaging device, contained within an external console, or may be a separate component.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An ultrasound imaging system comprising:
an array of acoustic elements configured to transmit ultrasound energy into a patient's anatomy and receive echoes associated with the transmitted ultrasound energy, wherein the acoustic elements receive the echoes according to a directivity of the acoustic elements; and
a processor in communication with the array and configured to:
receive, from the array, electrical signals corresponding to the received echoes;
generate an image based on the received electrical signals;
determine a portion of the received electrical signals corresponding to on-axis components, wherein the portion is determined based on the directivity of the acoustic elements and the received electrical signals;
generate a weighting mask based on the determined portion;
apply the weighting mask to the image to generate a grating lobe minimized image; and
output, to a display in communication with the processor, the grating lobe minimized image.

2. The ultrasound imaging system of claim 1, further comprising an intravascular ultrasound (IVUS) imaging catheter, wherein the array of acoustic elements is positioned around a circumference of the IVUS imaging catheter.

3. The ultrasound imaging system of claim 1, wherein the processor is configured to control the array by activating a plurality of apertures, and wherein the processor is configured to determine the portion of the received electrical signals corresponding to the on-axis components by:
determining an amplitude response of each aperture for the on-axis components of the received electrical signals; and
decomposing the amplitude of the electrical signals into a plurality of amplitude basis vectors, wherein the amplitude basis vectors are created based on the directivity of the acoustic elements.

4. The ultrasound imaging system of claim 1, wherein the processor is configured to determine the directivity of the acoustic elements based on an angular position of the acoustic elements in the array and an acceptance angle between the acoustic elements and a target position.

5. The ultrasound imaging system of claim 4, wherein the processor is configured to determine the directivity of the acoustic elements based further on ultrasound image data.

6. The ultrasound imaging system of claim 4, wherein the processor is configured to generate the weighting mask by generating a weight vector of M elements, wherein the M elements of the weight vector correspond to signal contributions from M different angles of arrival.

7. The ultrasound imaging system of claim 6, wherein the processor is configured to generate the weighting mask by identifying an element of the weight vector that corresponds to an angle of arrival of 0°.

8. The ultrasound imaging system of claim 1, wherein the processor is configured to apply the weighting mask to the image by multiplying the image with the weighting mask.

9. A method for ultrasound imaging, comprising:
controlling, by a processor, an array of acoustic elements in communication with the processor to transmit ultrasound energy into a patient's anatomy and receive echoes associated with the transmitted ultrasound energy, wherein the acoustic elements receive the echoes according to a directivity of the acoustic elements;
receiving, at the processor, electrical signals corresponding to the received echoes;
generating an image based on the received electrical signals;
determining a portion of the received electrical signals corresponding to on-axis components based on the directivity of the acoustic elements and the received electrical signals;
generating a weighting mask based on the determined portion;
applying the weighting mask to the image to generate a grating lobe minimized image; and
outputting, to a display in communication with the processor, the grating lobe minimized image.

10. The method of claim 9, wherein controlling the array to transmit ultrasound energy comprises controlling an intravascular ultrasound (IVUS) imaging catheter, wherein the array of acoustic elements is positioned around a circumference of the IVUS imaging catheter.

11. The method of claim 9, wherein controlling the array to transmit the ultrasound energy and to receive the echoes comprises activating a plurality of apertures, and wherein determining the portion of the received electrical signals corresponding to the on-axis components comprises:
determining an amplitude response of each aperture for the on-axis components of the received electrical signals; and
decomposing an amplitude of the electrical signals into a plurality of amplitude basis vectors, wherein the amplitude basis vectors are created based on the directivity of the acoustic elements.

12. The method of claim 9, wherein determining the directivity of the acoustic elements comprises determining the directivity of each acoustic element based on an angular position of the acoustic element in the array and an acceptance angle between the acoustic element and a target position.

13. The method of claim 12, wherein determining the directivity of the acoustic elements further comprises determining the directivity of the acoustic elements based on ultrasound image data.

14. The method of claim 12, wherein generating the weighting mask comprises generating a weight vector of M elements, wherein the M elements of the weight vector correspond to signal contributions from M different angles of arrival.

15. The method of claim 14, wherein generating the weighting mask further comprises identifying an element of the weight vector that corresponds to an angle of arrival of 0°.

16. The method of claim 9, wherein applying the weighting mask to the image comprises multiplying the image with the weighting mask.

17. An ultrasound imaging system, comprising:
an array of acoustic elements configured to transmit ultrasound energy into a patient's anatomy and receive echoes associated with the transmitted ultrasound energy; and
a processor in communication with the array and configured to:
generate image data corresponding to the received echoes;
generate an image based on the image data;
decompose the image data into a plurality of amplitude basis vectors corresponding to different angles of arrival;
generate a weighting mask based on the plurality of amplitude basis vectors;
generate a grating lobe minimized image by applying the weighting mask to the image; and
output, to a display in communication with the processor, the grating lobe minimized image.

18. The ultrasound imaging system of claim 17, wherein the processor is configured to generate the weighting mask by determining, based on the plurality of amplitude basis vectors, a portion of the image data corresponding to on-axis components, wherein the portion is determined based on a directivity of the acoustic elements of the array and the image data.

19. The ultrasound imaging system of claim 18, wherein the directivity of the acoustic elements is determined based on an angular position of the acoustic elements in the array and an acceptance angle between the acoustic elements and a target position.

20. The ultrasound imaging system of claim 19, wherein the directivity of the acoustic elements is determined based on ultrasound data.
